(19) United States
(12) Patent Application Publication
(10) Pub. No.: US 2016/0106421 A1
(43) Pub. Date: Apr. 21, 2016

(54) A SHAFT-PUSHED FASTENABLE SUTURE

(71) Applicant: KEREN MEDICAL LTD., Jerusalem (IL)

(72) Inventors: Eliahu ELIACHAR, Haifa (IL); Nir LILACH, Kfar Yehoshua (IL); Amir BARZILAY, Kokhav Yair (IL)

(73) Assignee: KEREN MEDICAL LTD., Jerusalem (IL)

(21) Appl. No.: 14/787,475

(22) PCT Filed: Apr. 28, 2014
(86) PCT No.: PCT/IL2014/050383

§ 371 Date: Oct. 28, 2015

Related U.S. Application Data
(60) Provisional application No. 61/816,831, filed on Apr. 29, 2013.

(51) Int. Cl. A61B 17/06; A61B 17/11; A61B 17/12; A61B 17/04

(52) U.S. Cl. CPC A61B 17/06166; A61B 17/0401; A61B 17/11; A61B 17/12009; A61B 17/06004; A61B 2017/06042; A61B 2017/06176; A61B 2017/1107

(57) ABSTRACT

A shaft-pushed fastenable suture configured for suturing a first tissue with a second tissue and/or for ligation of blood vessels and/or hollow structured organs. The fastenable suture characterized by an elongated flexible cord with one or more teeth, protruding from same. The fastenable suture further comprises a male-head at the distal end and an accepting female-head at the proximal end. The male-head is in connection with an anchor. The anchor configured to be temporarily accommodated by a distal end of a pushing-shaft. The female-head comprises a suture accepting-channel and a neighboring pushing-shaft accepting-channel. At least one of the two accepting-channels comprising one or more flexible pawls for both allowing, by means of the teeth, a continuous linear forward motion of the male-head and the cord within the accepting-channel in only one direction, while preventing motion of the male-head and cord in a backward direction and allowing only the pushing-shaft a reversible reciprocal motion.

### FIELD OF THE INVENTION

The present invention generally relates to a medical device for surgical procedures and specifically to tissue suturing with a fastenable suture.

### BACKGROUND OF THE INVENTION

Surgical procedures tend to minimize an incision in a body tissue by utilizing advanced technology medical devices. Consequently, alternative suturing procedures have to be applied for addressing new substantially further complicated conditions facing the suturing procedures. In the procedure of anastomosis of the urethra and the bladder during radical retro-pubic prostatectomy, for example, the attachment of the urethral stump to the bladder neck is particularly difficult and carried out semi-automatically by a surgical medical device.

The prior art of manual suturing cannot be applied in these surgical procedures without further abdominal cutting that provides an easier access to the tissues' area. This procedure has significantly high morbidity because of the presence of several large blood vessels in the operating region. It is not uncommon for the needles to slip or tear through these blood vessels and cause significant bleeding. Another disadvantage of the abdominal approach is the risk of punctures to the surgeon's finger; such punctures could lead to transmission of hepatitis, auto-immune deficiency virus (AIDS) and other serious diseases.

There is therefore a need to ensure a minimal tissues' puncture and no tissue damaged as the suture is drawn through. When using surgical needles, the size of the needle and suture should be carefully matched to ensure that the suture does not tear tissue and to prevent fluid leakage from the vessel. Accordingly, a need exists for a surgical needle and/or suture, which penetrates tissue in a less traumatic manner to facilitate healing.

Hulliger, US App. 2012/0289962, disclosed a system for securing a bone fixation device to a bone comprises a flexible longitudinal element configured to wrap about and stabilize one of a target portion of bone and a bone stabilizing element. The longitudinal element includes a plurality of projections extending distally from a first surface thereof. Each projection includes a proximal abutting surface in combination with a bone fixation element including a channel extending therethrough sized and shaped to slidably receive therein a portion of the longitudinal element distal of the head. The bone fixation element includes a recess shaped to receive the head and prevent the head from being drawn distally through the channel. The bone fixation element or the longitudinal element includes a tab configured to engage one of the proximal abutting surfaces to prevent the projections of the longitudinal element from moving proximally relative to the tab.

Browne, US App. 2012/0266419, disclosed active material based fasteners that are reversible. These fasteners can be in the form of, for example, cable ties and twist ties. In an embodiment, a fastener for securing an object comprises: a flexible elongated member capable of at least partially surrounding the object; and an adjustment mechanism in operable communication with the flexible elongated member, the adjustment mechanism comprising an active material capable of undergoing a change in a property upon exposure to an activation source, wherein the elongated member is configured to engage or release the object in response to the change in the property.

Golden, U.S. Pat. No. 6,695,859, disclosed an anastomosis system comprising: a tubular member having an end with an edge adapted to form an opening in a vessel wall; and an occlusion member adapted to be slidably coupled to the tubular member and adapted to substantially occlude the opening in the vessel wall to form an area of hemostasis, further comprising an anchor member adapted to hold the vessel wall in place while the tubular member is forming the opening in, the vessel wall, the anchor member being slidably coupled to the tubular member, wherein the anchor member comprises a shaft and a piercing member extending from a distal end of the shaft, the piercing member being adapted to pierce the vessel wall, further comprising a generally circular centering disk slidably movable along the shaft.

Browning, U.S. Pat. No. 8,182,413, disclosed a method for supporting a urethra of a patient includes making an incision on an upper wall of a vagina of the patient and inserting a first end of a surgical implant through the incision and on a first side of a urethra of the patient. The first end of the surgical implant has a first fixing zone with a retaining portion that is formed of a fiber entanglement and a mesh.

Lee, U.S. Pat. No. 8,177,836, disclosed a surgical fastening apparatus including at least one self-closing clip and a deployment device. The self-closing clip comprises a wire defining an intermediate portion interconnecting opposing, first and second side portions having a memory set loop shape. The deployment device includes a clip holding assembly and an actuator. The clip holding assembly includes first and second containment arms and a transfer rod. The containment arms have a distal segment defining a lumen extending from an open, distal end. The transfer rod is associated with at least one of the containment arms in an axially movable fashion and forms an engagement feature. The actuator is connected to the transfer rod for controlling movement of the rod. Upon final assembly in a pre-deployment state, the engagement feature of the transfer rod engages the intermediate portion of the clip, and the side portions are captured by the containment arms.

Schaller et al, U.S. Pat. No. 8,394,114, disclosed surgical connection apparatus which comprises a support structure, a plurality of clips, which can be self-closing clips, each clip being releasably coupled to the support structure, and a plurality of barbs, each barb being coupled to the support structure, the barbs being separate from the clips, which are ejectable from the support structure independently of the barbs.

Apparently, in light of the prior art, there is still a long felt need for a fastenable suture configured for soft tissue suturing with a minimal tissue puncture area, while avoiding the unnecessary rupturing and tearing of the penetrated tissues and the rapture of the adjacent blood vessels.

Suturing inside the body, especially when it is a part of laparoscopic or robotic surgery is complex and challenging for the following main reasons:
The suture is performed from a distance of several centimeters to several tens of centimeters inside the body, which means that it can only be performed with surgical tools. Surgical tools limit the sensation of the tissue(s) being sutured. In robotic surgery there is no tissue sensing at all.
The laparoscopic and robotic arms use minimal invasive ports as an “axis” for the movement of the tool(s). Thus, the surgeon needs to move his/her arms in the opposite direction in order to perform a surgical action in the desired direction, which is contra-intuitive.
Suturing is primarily performed by a combined movement of the wrist and elbow. Unfortunately, from an anatomic point of view, it is limited in angle-of-movement, which is partially resolved in the robotic surgery.
Anastomosis is the one of the most challenging areas of suturing, because it can cause one or both of the following: (a) leakage of fluids inside the body (b) block of flow from between the two anastomosed organs. Anastomosis becomes even more complex and challenging where: (a) the diameter of the anastomosed organs is not equal (b) at least one of the organs is very small in diameter, e.g. under 20 mm.
Frequently, suturing or anastomosis requires access to locations which are difficult to access due to being “under” organs which cannot be temporarily moved, lifted or twisted for suturing, or due to a limited space. Examples for such cases include anastomosis of the colon, the small intestines, esophageal anastomosis and bladder-urethra anastomosis.
Frequently, especially in laparoscopic and robotic surgery, the tissues which need to be anastomosed are not uniform in shape and thickness. This requires extra caution and technique of the surgeon to succeed in the suturing or anastomosis.

As a result of the mentioned above, automation of surgical suturing, and to a greater extent, automation of surgical anastomosis is still very limited. It still takes a significant amount of surgeon experience, surgery and operating room time to complete inside body suturing and to a greater extent, multiple organ anastomosis.

Today, the state-of-the-art devices for reducing suturing complexity and time are still, in practice manual suturing devices. For example, two advanced suturing devices are: SILS™ by Covidien and Endo360™ by EndoEvolution.

The SILS™ stitch device enables the automatic transfer of a straight (rather than curved) surgical needle from one “jaw” of the device to the other “jaw”, yet, the remaining surgical actions by the surgeon, especially the complex suture generation, remains completely manual.

The main advantages of the Endo360™ over the SILS™ solution, lies in its curved needle design and its ability to fully rotate the needle with the suture thread 260 degrees, thus further automating the traditional fully manual suture thread positioning and passing through the tissue(s). Yet, tying and fastening and suture thread with the utilization of the Endo360™ solution remains completely manual. Where a significant number of sutures is required, especially in the event of end-to-end and end-to-side anastomosis, solutions such as SILS™ and Endo360™ provide little help in successfully and safely performing the task.

Another example of a common device used for end-to-end anastomosis is circular stapler, such as the DST™ stapler by Covidien.

Circular staplers are typically used to anastomose the remaining parts of the organs most frequently resected as a result of cancer. Mostly, such devices are for colon anastomosis following partial colon resection as a result of colon cancer. The device first “folds” inside the boundaries of the resected colon tissue and then delivers several circles of staples to anastomose both ends of the remaining colon parts. The anastomosis process mandates the reduction of the original diameter of the colon, creates an inner scar or bulb tissue within the colon and reduces the amount of flow between the two anastomosed colon portions. To date, the minimum diameter which can be handled by such devices, due to the inner tissue “folding”, is 21 mm. Further, circular staplers are designed for end-to-end anastomosis of resected organs, where the diameter of the parts being anastomosed is practically identical. In the event that the anastomosed organ diameter is smaller than 21 mm and where the diameter of the anastomosed organs is significantly different, such circular stapler technology is of no use.

An example for anastomosis, which requires significant surgeon skills and operating room time, is the anastomosis of the remains of the bladder neck and the remains of the urethra, following Radical Prostatectomy, a surgery involving the complete resection of the Prostate Gland as a result of Prostate Cancer.

In order to refrain from leaving cancerous Prostate Cancer tissues or cells, the surgeon, with the purpose of leaving “safety margins”, resects the bladder neck proximally to the Bladder-Prostate junction and also resects the Prostate-Urethra junction distally to the Prostate-Urethra junction. The outcome of such resection are an enlarged Bladder neck with an oval shape and a typical diameter of 30-45 mm, and a very short remains of the Urethra, typically 5-10 mm long, with an inner diameter of 7-8 mm. Further, due to the fact that the resection is performed indirectly through electrical energy, the tissue margins of the remaining oval Bladder neck and the circular Urethra are far from uniform, further making the anastomosis challenging and complex to perform. In addition to all that, in a laparoscopic or robotic surgery, the surgical ports are made in the center of the abdomen, while the surgery takes place over 50 cm distally to the ports, making any surgical action even more challenging.

Today, in both laparoscopic and robotic surgery, the anastomosis of the remaining Bladder neck and the remaining Urethral stump is performed manually. Since the diameter of the Bladder neck and the Urethra are so significantly different (30-45 mm vs. 7-8 mm), the surgeon has no other choice but to suture the Urethra either to the right side of the Bladder neck or to the left side of the Bladder neck.

Using either conventional or barbed suture threads, the surgeon first creates a first suture at the 6 o'clock location, otherwise, there will be no access to this location. Then, the surgeon continues with a suture at 5 o'clock, followed by a suture at 7 o'clock, and so forth, until the 8 mm diameter of the Urethra are anastomosed to a partial portion of the Bladder neck. Because sutured tissues are far from uniform, each suture needs to be placed very carefully and tested for being sufficiently strong not to cut through the tissue and become loose. To complete the anastomosis, the surgeon now sutures the remaining (still open) Bladder neck with another significant number of sutures.

A proficient laparoscopic or robotic surgeon will need 15-25 minutes to successfully complete such anastomosis. A less proficient surgeon will require 40-90 minutes to complete the task. In about 10%-15% of the Bladder-Urethra anastomosis cases, the patients suffer either a blockage of the anastomosed Bladder-Urethra passage, or from urine leaks into the abdominal space, both are severe side effects.

In light of all the mentioned above, there is a strong need for a device and method, which (a) reduce the surgeon proficiency requirements; (b) reduce the anastomosis complexity; (c) reduce the surgery time; and improve the outcomes and reduce side effects.

### SUMMARY OF THE INVENTION

It is one object of the present invention to disclose a shaft-pushed fastenable suture (100) for suturing a first tissue with a second tissue, characterized by an elongated flexible cord (5), with one or more teeth (6) protruding from same, having a male-head (1) at the distal end and an accepting female-head (2) at the proximal end; the male-head (1) is in connection with an anchor (3); the anchor configured to be temporarily accommodated by a distal end (4b) of a pushing-shaft (4); the female-head (2) comprising a suture accepting-channel (2a) and a neighboring pushing-shaft accepting-channel (2b); at least one of the two accepting-channels (2a, 2b) comprising one or more flexible pawls (2c) for both allowing, by means of the teeth (6), a continuous linear forward motion of the male-head (1) and the cord (5) within the accepting-channel (2a, 2b) in only one direction, while preventing motion of the male-head (1) and cord (5) in a backward direction and allowing only the pushing-shaft (4) a reversible reciprocal motion.

It is another object of the present invention to disclose a shaft-pushed fastenable suture (100) for ligation of at least one blood vessels and/or hollow structured organ, characterized by an elongated flexible cord (5), with one or more teeth (6) protruding from same, having a male-head (1) at the distal end and an accepting female-head (2) at the proximal end; the male-head (1) is in connection with an anchor (3); the anchor configured to be temporarily accommodated by a distal end (4b) of a pushing-shaft (4); the female-head (2) comprising a suture accepting-channel (2a) and a neighboring pushing-shaft accepting-channel (2b); at least one of the two accepting-channels (2a, 2b) comprising one or more flexible pawls (2c) for both allowing, by means of the teeth (6), a continuous linear forward motion of the male-head (1) and the cord (5) within the accepting-channel (2a, 2b) in only one direction, while preventing motion of the male-head (1) and cord (5) in a backward direction and allowing only the pushing-shaft (4) a reversible reciprocal motion.

It is another object of the present invention to disclose the fastenable suture (100) as defined above, wherein the cord (5) having a crescent-like (5a) cross-section (A-A), configured to be at least partially attached with the pushing-shaft (4), for a small puncture and a smooth passage through the first and second tissues.

It is another object of the present invention to disclose the fastenable suture (100) as defined above, wherein the pushing-shaft (4) having a crescent-like (4a) cross-section (B-B) configured to be at least partially attached with the cord (5), for a small puncture and a smooth passage through the first and second tissues.

It is another object of the present invention to disclose the fastenable suture (100) as defined above, wherein the one or more teeth (6) having a conic structure (6a), starting from the distal edge (6b) of the teeth (6) and expanding towards the proximal edge (6c) of same, configured for a smooth passage through the first and second tissues and for preventing the backward motion through the suture accepting-channel (2a) such that the first and second tissues are protected from being ruptured or torn by the teeth (6).

It is another object of the present invention to disclose the fastenable suture (100) as defined above, wherein the male-head (1) comprises a sharp tip (1a) configured to be pushed by the pushing-shaft (4) through the first and second tissues.

It is another object of the present invention to disclose the fastenable suture (100) as defined above, wherein the female-head (2) configuration together with the pushing-shaft distal end (4b) are adapted to create tensile-tension to the first and second tissues, as the pushing-shaft distal end (4b) approaches the female-head (2), such that the friction of the suture (100) and the pushing-shaft (4) with the first and second tissues is reduced, a minimal puncture is obtained and tears, ruptures or organ blockage are avoided.

It is another object of the present invention to disclose the fastenable suture (100) as defined above, wherein the pushing-shaft distal end (4b) is of blunt contour (4i). It is another object of the present invention to disclose the fastenable suture (100) as defined above, wherein the suture accepting-channel (2a) and the neighboring pushing-shaft accepting-channel (2b) having a conic structure (2i, 2j), configured to centralize the approaching the male-head (1) and the distal end (4b) of the pushing-shaft (4) into the female-head (2).

It is another object of the present invention to disclose the fastenable suture (100) as defined above, wherein the one or more flexible pawls (2c) are configured by at least one notch (2d) passing through the conic suture accepting-channel (2i).

It is another object of the present invention to disclose the fastenable suture (100) as defined above, wherein the one or more flexible pawls (2c) are configured as a flexible flap-section (2e) having at least one line-notch (21); the flap-section (2e) is in connection with the female-head (2), configured to flip and fold toward the female-head (2), such that the pushing-shaft (4) and the suture (100) pass through both the accepting channels (2a, 2b) and the flap-section (2e).

It is another object of the present invention to disclose the fastenable suture (100) as defined above, wherein the flap-section (2e) further comprising a round-notch (2h) configured for the passage of the pushing-shaft (4).

It is another object of the present invention to disclose the fastenable suture (100) as defined above, wherein the accepting-channels (2a, 2b) are joined to a single channel (2f).

It is another object of the present invention to disclose the fastenable suture (100) as defined above, wherein the female-head (2) further comprising a detachable protecting-cap (7) at the outlet (2k) of the pushing-shaft accepting-channel (2b), configured to cover at least one selected from a group consisting of: the distal end (4b) of the pushing-shaft (4) and the male head (1), when passing through same, such that any other tissues and adjacent blood vessels are protected from being ruptured or torn.

It is another object of the present invention to disclose the fastenable suture (100) as defined above, wherein the protecting-cap (7) is temporarily attached to the outlet (2k) of the pushing-shaft accepting-channel (2b), by pressure-fasteners (7a) or by a connecting tear-strip between the outlet (2k) and the protecting-cap (7).

It is another object of the present invention to disclose the fastenable suture (100) as defined above, wherein the pushing-shaft (4) distal end (4b) comprising a sharp portion (4c) having an open bore (4d); the sharp portion (4c) comprises: a tip (4f) and a single notch (4g); the notch (4g) confined so as to be located on the open bore (4d), opposite to the tip (4f); the bore (4d) and the notch (4g) are adapted to accommodate the anchor (3).

It is another object of the present invention to disclose the fastenable suture (100) as defined above, wherein the dimensions of the anchor (3) are greater than the dimensions of the notch (4g), such that the anchor (3) is retained by the notch (4g) inside the open bore (4d), so as to prevent movement of the suture (100) relative to the pushing shaft (4).

It is another object of the present invention to disclose the fastenable suture (100) as defined above, wherein the shaft-pushed fastenable suture (100) is manufactured as one piece, such that the flexible flap-section (2e) is configured to flip and fold toward the female-head (2).

It is another object of the present invention to disclose the fastenable suture (100) as defined above, wherein the shaft-pushed fastenable suture (100) is manufactured as one piece, such that the protecting-cap (7) is configured to flip and fold toward the outlet (2k) of the female-head (2).

It is another object of the present invention to disclose the fastenable suture (100) as defined above, wherein the pushing-shaft (4) comprises a disengaging-rod (4h) threaded through the center axis of the pushing-shaft (4) and configured for relative motion inside the pushing-shaft (4) for separating and disengaging the anchor (3) from the pushing shaft (4) and releasing the fastenable suture (100) from the pushing-shaft (4).

It is another object of the present invention to disclose the fastenable suture (100) as defined above, wherein the anchor (3) comprises a rod-bore (3a) configured to temporarily accommodate the disengaging-rod (4h).

It is another object of the present invention to disclose the fastenable suture (100) as defined above, wherein the flexible flap-section (2e) further comprising at least one pin-notch (2p) and wherein the protecting cap (7) comprises at least one pin (7c), configured to be accommodated by the at least one of the pin-notches (2p).

It is another object of the present invention to disclose the fastenable suture (100) as defined above, wherein the suture's cord (5) comprises a weakened section (5b), which is narrower than the cord (5), located between the anchor (3) and the first adjacent the tooth (6), configured to be ripped and detached form the anchor (3), when the cord's (5) tensile tension has reached a predetermined tensile-tension.

It is another object of the present invention to disclose the fastenable suture (100) as defined above, wherein the fastenable suture (100) is configured for self-tying and self-fastening facilitating a fully automatic suturing.

It is another object of the present invention to disclose the fastenable suture (100) as defined above, wherein at least two of the fastenable sutures (100) and their accompanying the pushing shafts (4) are configured to be mounted on an automatic surgical suturing device.

It is another object of the present invention to disclose the fastenable suture (100) as defined above, wherein at least one of the one or more teeth (6) comprises at least one protruding flapping-tip (6d), at the proximal edge (6c).

It is another object of the present invention to disclose the fastenable suture (100) as defined above, wherein the at least one protruding flapping-tip (6d), is at least partially flexible.

It is another object of the present invention to disclose the fastenable suture (100) as defined above, wherein the at least one protruding flapping-tip (6d), is revolving toward and away from the cord (5).

It is another object of the present invention to disclose the fastenable suture (100) as defined above, wherein at least one of the one or more teeth (6) comprises a flap (6e), at the proximal edge (6c).

It is another object of the present invention to disclose the fastenable suture (100) as defined above, wherein the flap (6e), is at least partially flexible.

It is another object of the present invention to disclose the fastenable suture (100) as defined above, wherein the cord (5) comprises one or more backward facing barbs (6f).

It is another object of the present invention to disclose the fastenable suture (100) as defined above, wherein the one or more teeth (6) are configured as backward facing barbs (6f).

It is another object of the present invention to disclose the fastenable suture (100) as defined above, wherein the barbs (6f) are at least partially flexible.

It is another object of the present invention to disclose the fastenable suture (100) as defined above, wherein the barbs (6f) are evenly spread.

It is another object of the present invention to disclose the fastenable suture (100) as defined above, wherein the barbs (6f) are circumferentially distributed.

It is another object of the present invention to disclose the fastenable suture (100) as defined above, wherein the barbs (6f) are helically distributed.

It is another object of the present invention to disclose the fastenable suture (100) as defined above, wherein the suture (100) further configured for ligation of at least two blood vessels.

It is another object of the present invention to disclose the fastenable suture (100) as defined above, wherein the suture (100) further configured for ligation and at least partial occlusion of at least one blood vessel and/or hollow structured organ.

### BRIEF DESCRIPTION OF THE DRAWINGS

The invention is herein described, by way of example only, with reference to the accompanying drawings, wherein:

FIG. 1 is a schematic drawing of the shaft-pushed fastenable suture (100) and the pushing-shaft (4), describing the various components of same;

FIG. 2 is a schematic drawing of the fastenable suture (100) connected to the pushing-shaft (4), where the suture's male-head (1) is approaching the suture's female-head (2);

FIG. 3 is a schematic drawing of the fastenable suture (100) connected to the pushing-shaft (4), where the suture's male-head (1) and the suture's cord (5) are passing within the suture's female-head (2);

FIG. 4 is a schematic drawing of the distal end (4b) of the pushing-shaft (4) comprising a sharp portion (4c) having an open bore (4d), a tip (4f) and a single notch (4g);

FIGS. 5A and 5B are schematic drawings of the suture's cord (5) having a crescent-like cross-section (5a);

FIGS. 6A and 6B are schematic drawings of the pushing-shaft (4) having a crescent-like cross-section (4a);

FIGS. 7A and 7B are schematic drawings of the pushing-shaft (4) and the suture's cord (5) both having crescent-like cross-section (4a, 5a);

FIGS. 8A and 8B are schematic drawings the suture's teeth (6) having conic structure (6a);

FIG. 9 is a schematic drawing of another embodiment for the shaft-pushed fastenable suture (100) comprising: a cord (5), a male-head (1) and a female-head (2) having conic accepting channels (2i, 2j);

FIGS. 10A and 10B are schematic top and side views of the shaft-pushed fastenable suture (100), respectively, comprising the conic accepting channels (2i, 2j);

FIGS. 11A, 11B, 11C, 11D and 11E are schematic drawings of the fastenable suture (100), comprising the conic accepting channels (2i, 2j), being pushed by the pushing-shaft (4), shown in five stages: approaching, entering, fastening, retracing and detaching, respectively;

FIG. 12 is a schematic drawing of the suture's female-head (2), where the suture's accepting-channel (2a, 2b) have a conic structure (2i, 2j), and where the suture accepting-channel (2i) comprises notches (2d) passing through same;

FIG. 13 is a schematic drawing of the suture's female-head (2) further comprising a detachable protecting-cap (7) in connection with the outlet (2k) of the female-head (2) pushing-shaft's accepting-channel (2b);

FIG. 14 is a schematic side view of the fastenable suture (100), comprising the detachable protecting-cap (7);

FIGS. 15A, 15B and 15C are three schematic demonstrations of the pushing-shaft (4) pushing the protecting-cap (7), fastening the first sutures tooth (6) and retracing, respectively;

FIGS. 16A, 16B and 16C are schematic drawings of another embodiment for the fastenable suture (100), where the suture's female-head (2) is in connection with a flexible flap-section (2e), shown in two positions: open as in FIG. 16A and flipped toward the female-head (2), as in FIG. 16B top view and 16C bottom view;

FIGS. 17A and 17B are schematic demonstrations of the pushing-shaft (4) pushing the suture's male-head (1) towards, as in FIG. 17A, and into, as in FIG. 17B, the closed flexible flap-section (2e);

FIGS. 18A and 18B are two schematic views of another embodiment for the detachable protecting-cap (7) having two pins (7c), configured to accommodated by the pin-notches (2p) of the flexible flap-section (2e);

FIGS. 19A, 19B and 19C are schematic demonstrations of the pushing-shaft (4) pushing the male-head (1) into the female-head (2) having the protecting-cap (7) with the two pins (7c), shown in three stages: entering the female-head (2), pushing out the protecting-cap (7) and retracting, respectively;

FIG. 20 is a schematic drawing of another embodiment of the fastenable suture (100), where the cord (5) comprises a weakened section (5b), configured for detaching the anchor (3);

FIGS. 21A and 21B are schematic drawings of another embodiment of the suture's teeth (6), having at least one protruding flapping-tip (6d);

FIGS. 22A and 22B are schematic drawings of another embodiment of the suture's teeth (6), having a flexible flap (6e);

FIGS. 23A, 23B and 23C are schematic drawings of another embodiment of the suture's cord (5), having at least one backward facing barb (6f); and

FIGS. 24A, 24B and 24C are schematic drawings of another embodiment of the one or more teeth (6) configured as backward facing barb (6f).

### DETAILED DESCRIPTION OF THE PREFERRED EMBODIMENT

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a device for a full automatic suturing of a first tissue with a second tissue, such as intra-luminal, lumen-to-lumen and side-to-lumen and for the suturing of: (a) small diameter to larger diameter organs (b) small diameter to small diameter organs.

The term “suture”, used herein, refers to a strand or fiber used to sew parts of the living body; it can also refer to a ligature, such as a filament or thread, used to unite, connect, bind or tie blood vessels and/or hollow structured organs.

The present invention is a new shaft-pushed fastenable suture (100) for suturing a first tissue with a second tissue, characterized by an elongated flexible cord (5). The cord (5) comprises one or more teeth (6) protruding from same. The cord (5) further comprises a male-head (1) at the distal end and an accepting female-head (2) at the proximal end. The male-head (1) is in connection with an anchor (3). The anchor configured to be temporarily accommodated by a distal end (4b) of a pushing-shaft (4). The female-head (2) comprising a suture male-head accepting-channel (2a) and a neighboring pushing-shaft accepting-channel (2b). At least one of the two accepting-channels (2a, 2b) comprising one or more flexible pawls (2c) for both allowing, by means of the teeth (6), a continuous linear forward motion of the cord (5) within the accepting-channels (2a, 2b) in only one direction, while preventing motion in a backward direction and allowing only the pushing-shaft (4) a reversible reciprocal motion.

The present invention further provides a shaft-pushed fastenable suture (100) for ligation of at least one blood vessels and/or hollow structured organ, characterized by an elongated flexible cord (5), with one or more teeth (6) protruding from same, having a male-head (1) at the distal end and an accepting female-head (2) at the proximal end; the male-head (1) is in connection with an anchor (3); the anchor configured to be temporarily accommodated by a distal end (4b) of a pushing-shaft (4); the female-head (2) comprising a suture accepting-channel (2a) and a neighboring pushing-shaft accepting-channel (2b); at least one of the two accepting-channels (2a, 2b) comprising one or more flexible pawls (2c) for both allowing, by means of the teeth (6), a continuous linear forward motion of the male-head (1) and the cord (5) within the accepting-channel (2a, 2b) in only one direction, while preventing motion of the male-head (1) and cord (5) in a backward direction and allowing only the pushing-shaft (4) a reversible reciprocal motion.

The present invention is a combination of a self-tying and self-fastening suture, combined with a delivery and release pushing shaft (4) complex, facilitating the fully automatic suturing and/or anastomosis of similar-in-diameter or different-in-diameter organs or tissues, frequently with asymmetric tissue margins, where the entire process is either performed from within the anastomosed organs, from outside the anastomosed organs, or any combination thereof.

The present invention is a pushing-shaft (4) delivered suture (100), which is based on the following innovative principles:
the suture area is small, causing minimal healthy tissue damage;
the pushing-shaft (4) and suture (100) area together are small, causing minimal healthy tissue damage;
the suture (100) tying is fully automatic, requiring no surgeon skills and/or intervention;
the suture (100) fastening is fully automatic and the suture fastening force is pre-defined and/or controllable, requiring no surgeon skills and/or intervention;
the pushing-shaft (4) and suture (100) dimensions are sufficiently small, enabling its delivery from within the anastomosed organ(s), even where the organ is of a small diameter;
the concurrent delivery of multiple sutures (100) is also supported, resolving the following problems, difficulties and challenges:
   anastomosed margins absence of uniformity,
   differences in anastomosed organs diameters, and
   access to inaccessible anastomosis locations;
the suture supports tying and fastening from inside anastomosed organs;
the pushing-shaft (4)—suture (100) combination facilitates, with a single continuous action, the delivery of the suture through first anastomosed tissue, the delivery of the suture (100) through second anastomosed tissue, the insertion of the male head (1) of the suture (100) through the female head (2) and the fastening the suture (100);
when the suturing is performed from within the anastomosed organ/s, the risk of organ blockage is removed.

In the specific example of Bladder-Urethra anastomosis, the current completely manual and tedious suturing, as in the prior art, is performed using laparoscopic arms through the abdominal ports in the abdominal space outside the anastomosed Bladder and Urethra. The disclosed invention, using a plurality of pushing-shaft (4) delivered sutures (100), enables the delivery of the sutures from within the Urethra. The needle-pushed sutures pass from within the Urethra outside, directly into the Bladder wall, and after passing through the Bladder wall, the needles concurrently insert the suture male (1) portions accurately into its counterpart female portions, pushes the male portions forward until each suture separately is sufficiently fastened with no possibility of loosening, and then, the pushing-shafts retract backward, leaving the Bladder neck and Urethra tightly anastomosed with the plurality of fastened sutures (100).

Before explaining the figures, it should be understood that the invention is not limited in its application to the details of construction and the arrangement of the components set forth in the following description or illustrated in the drawings. The invention can be carried out in various ways.

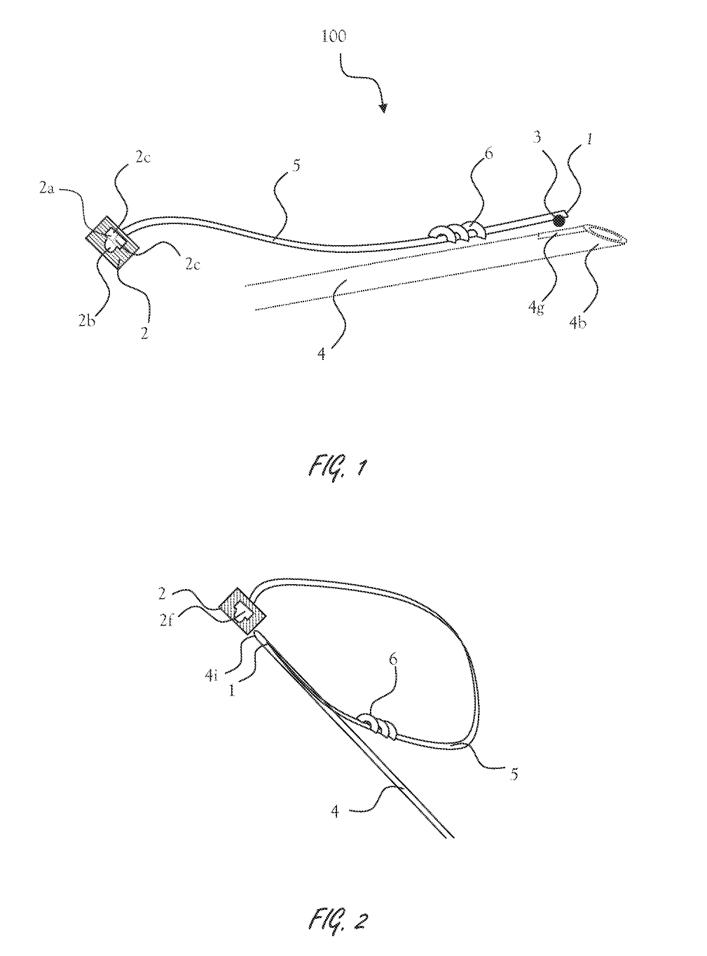

Reference is now made to FIG. 1 which is a schematic view of the shaft-pushed fastenable suture (100) and the pushing-shaft (4), describing the various components of same. The shaft-pushed fastenable suture (100) comprises an elongated flexible cord (5), with one or more teeth (6) protruding from same, a male-head (1) at the distal end of the cord (5) and an accepting female-head (2) at the proximal end. The male-head (1) is in connection with an anchor (3). The anchor configured to be temporarily accommodated by a distal end (4b) of the pushing-shaft (4). The female-head (2) comprising a suture accepting-channel (2a) and a neighboring pushing-shaft accepting-channel (2b). The suture accepting-channel (2a) comprises one or more flexible pawls (2c) for both allowing, by means of the teeth (6), a continuous linear forward motion of the male-head (1) and the cord (5) within the suture accepting-channel (2a) in only one direction, while preventing the suture's (100) male-head (1) and cord (5) motion in a backward direction and allowing only the pushing-shaft (4) a reversible reciprocal motion.

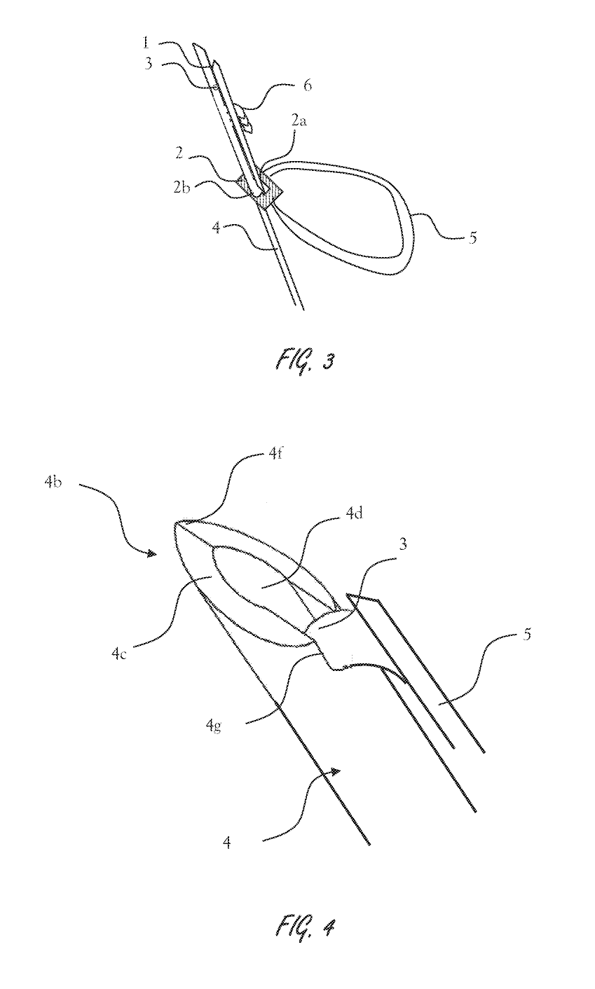

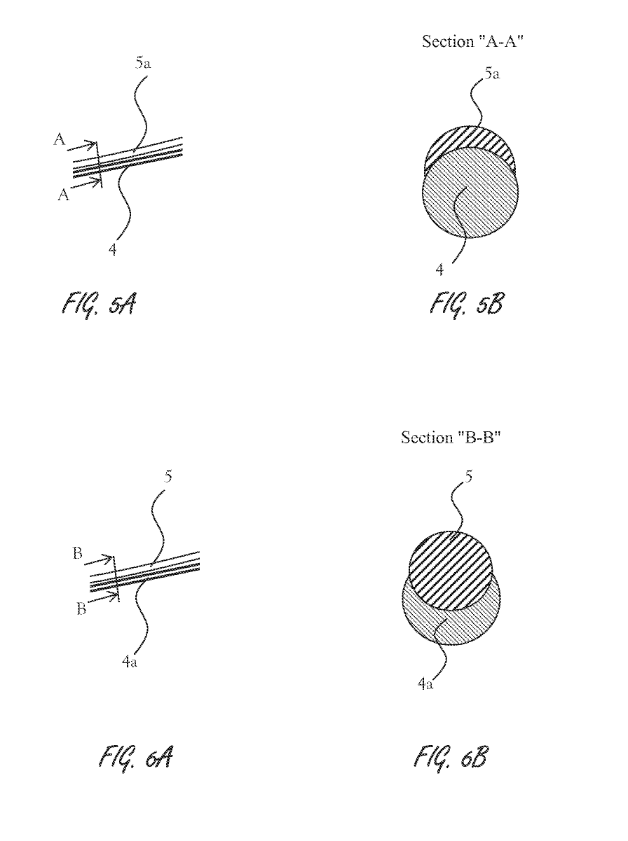

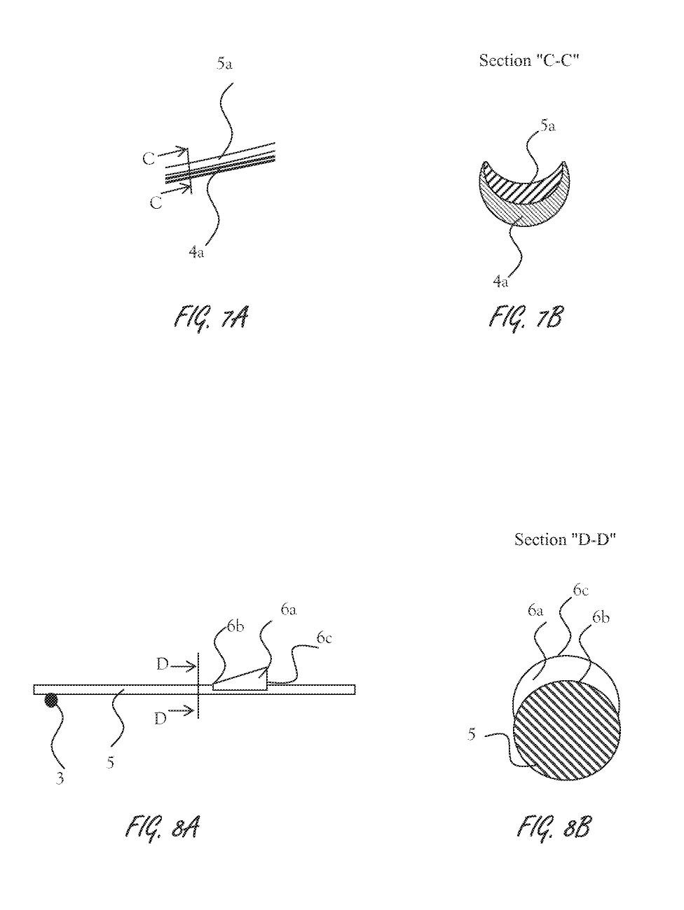

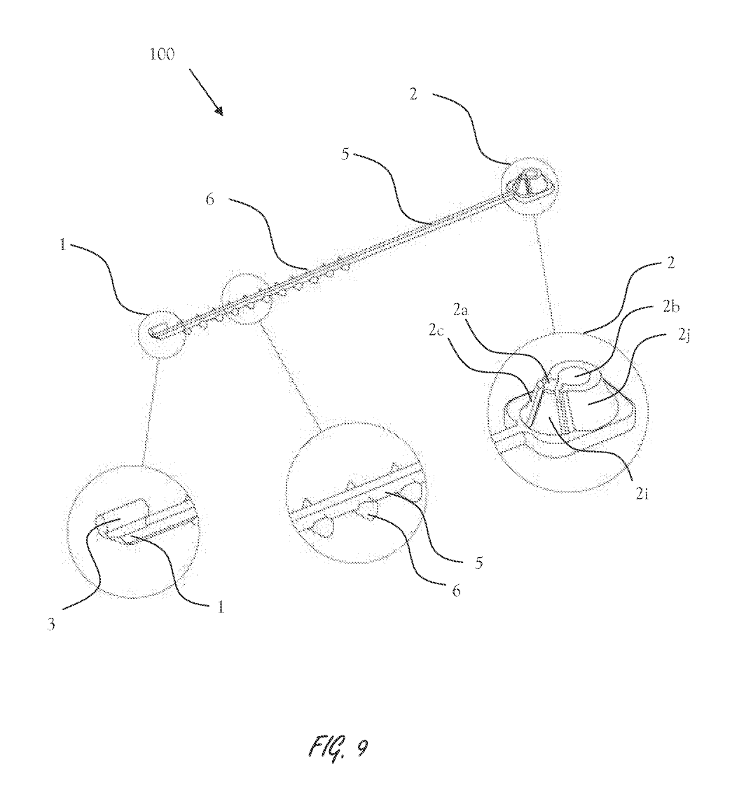

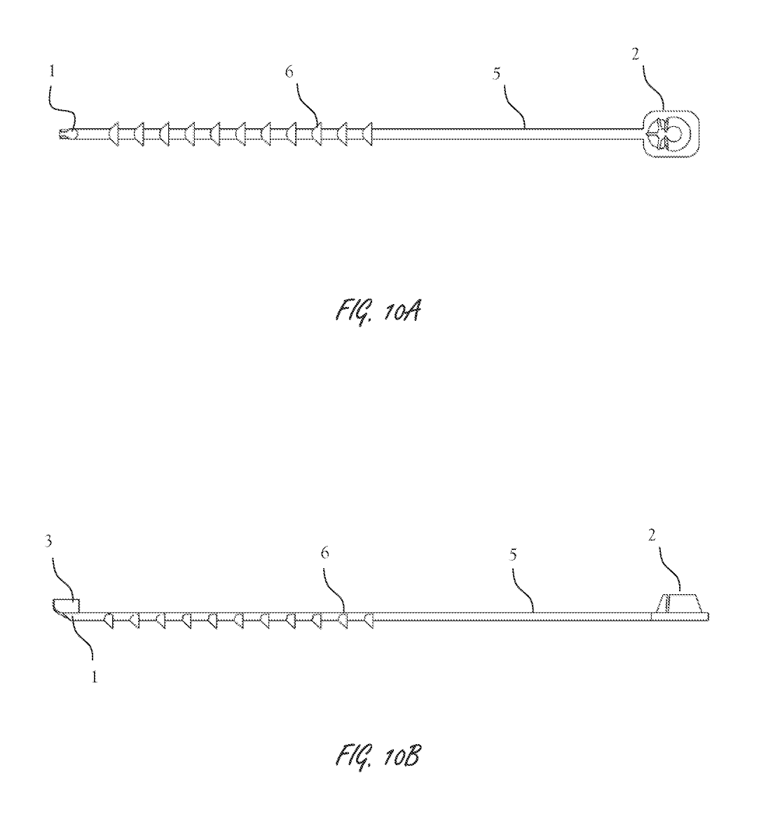

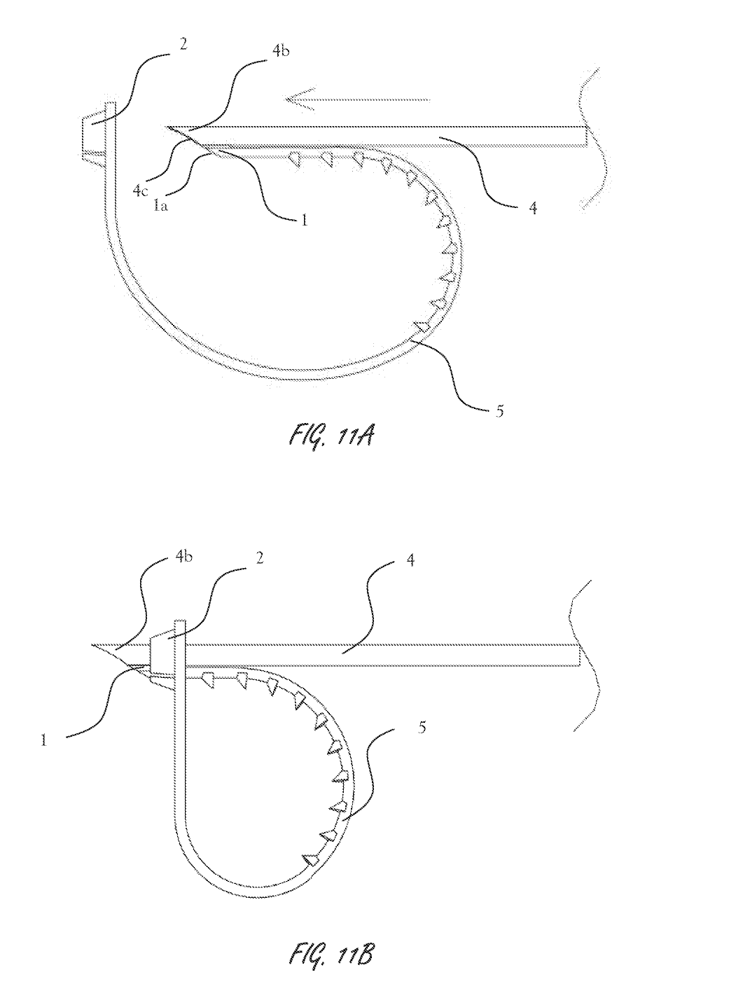

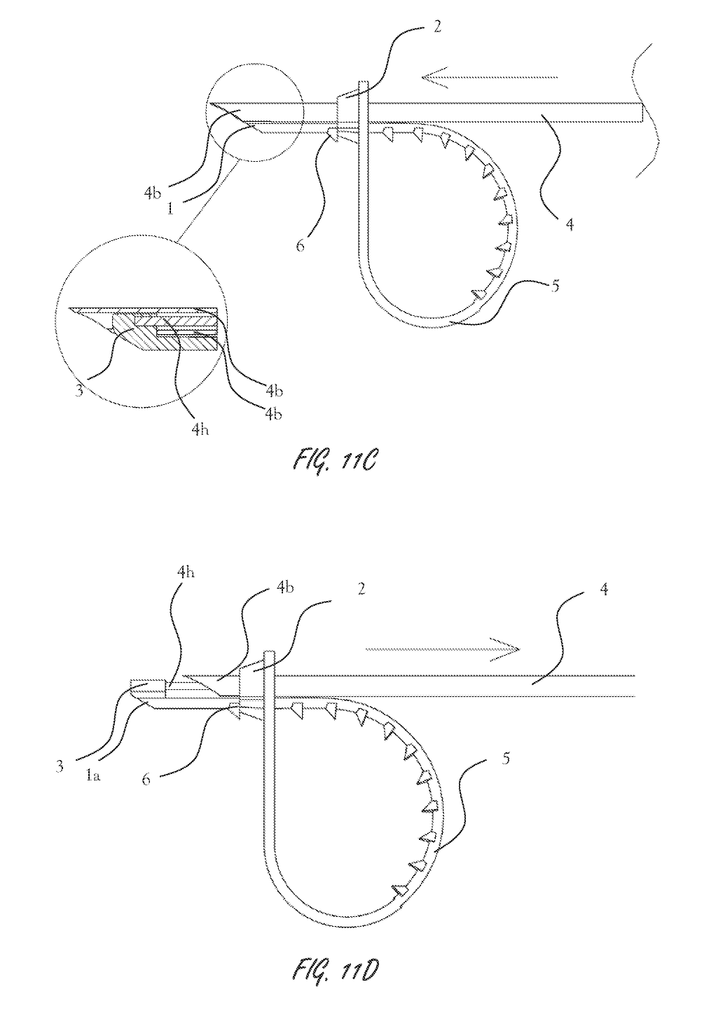

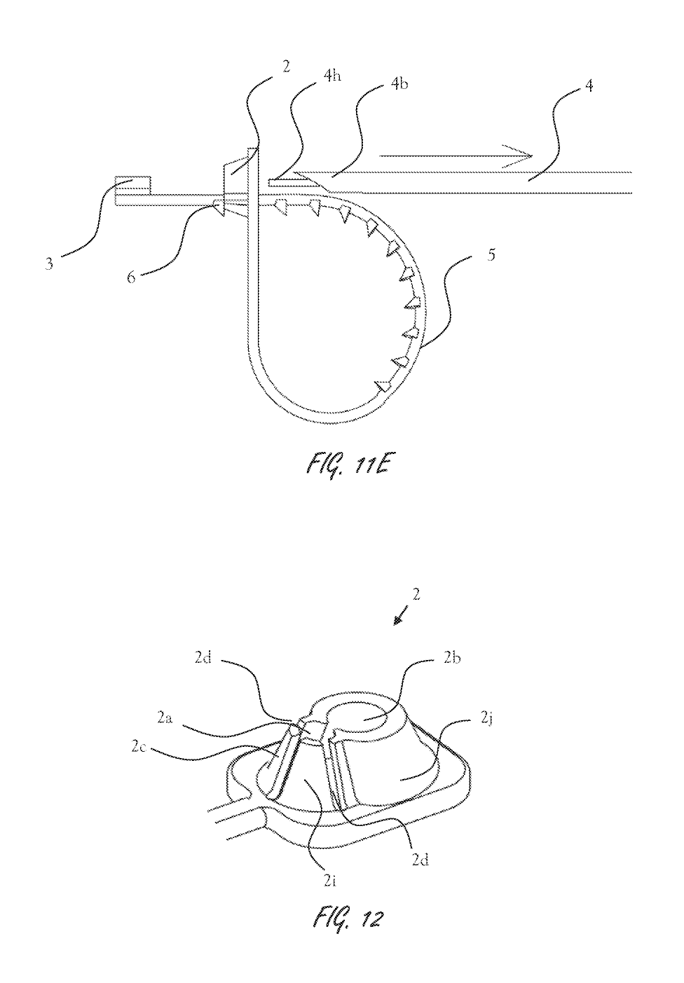

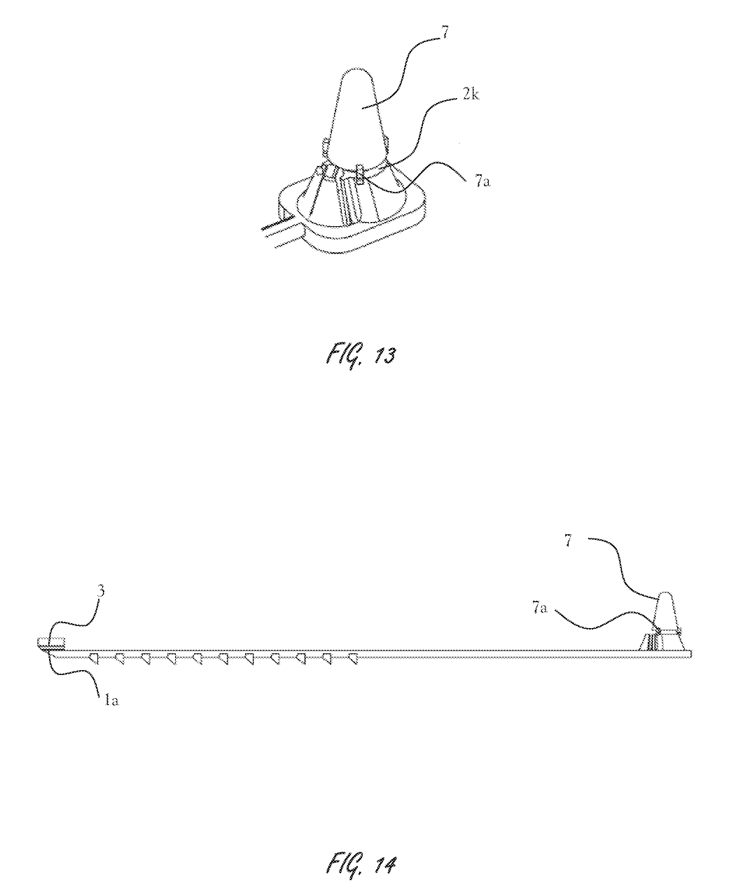

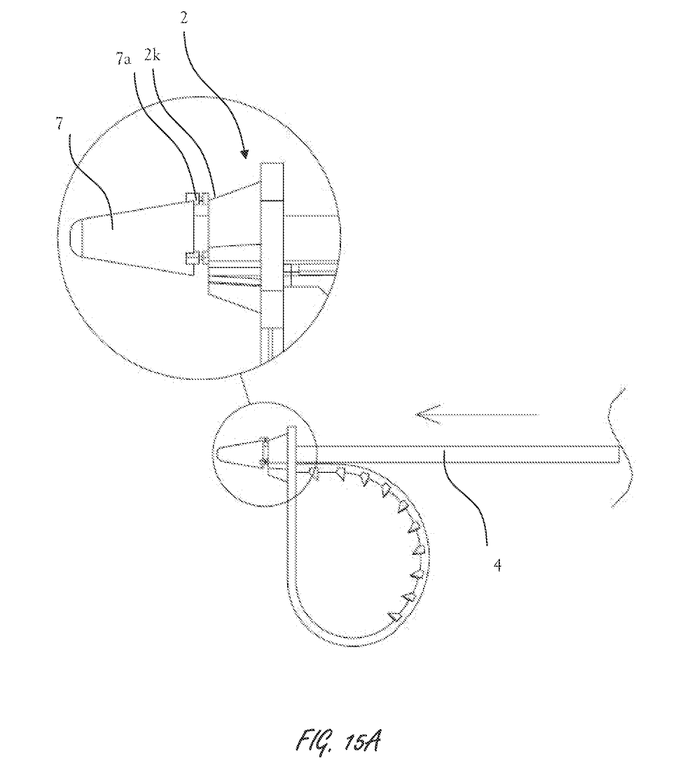

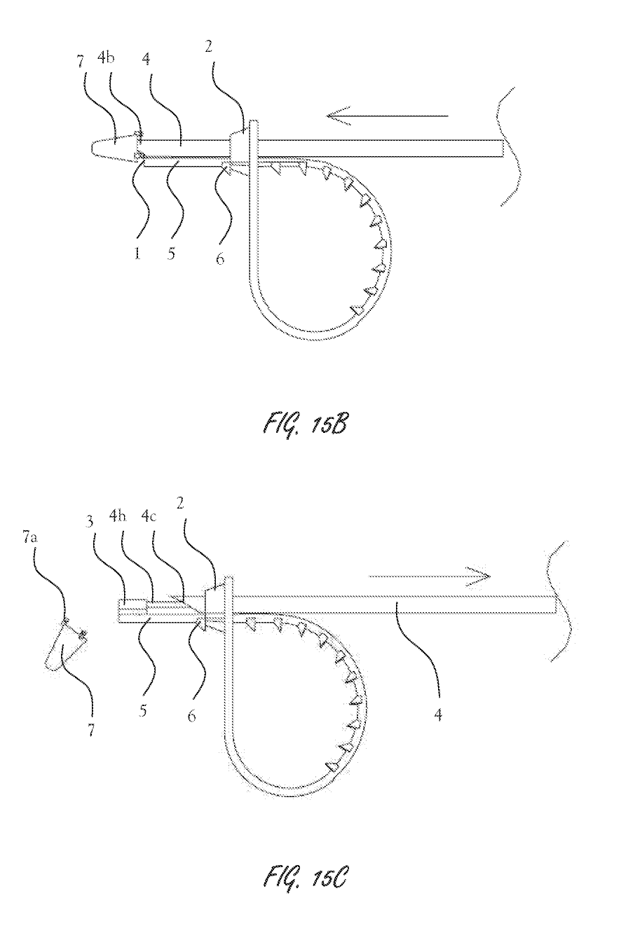

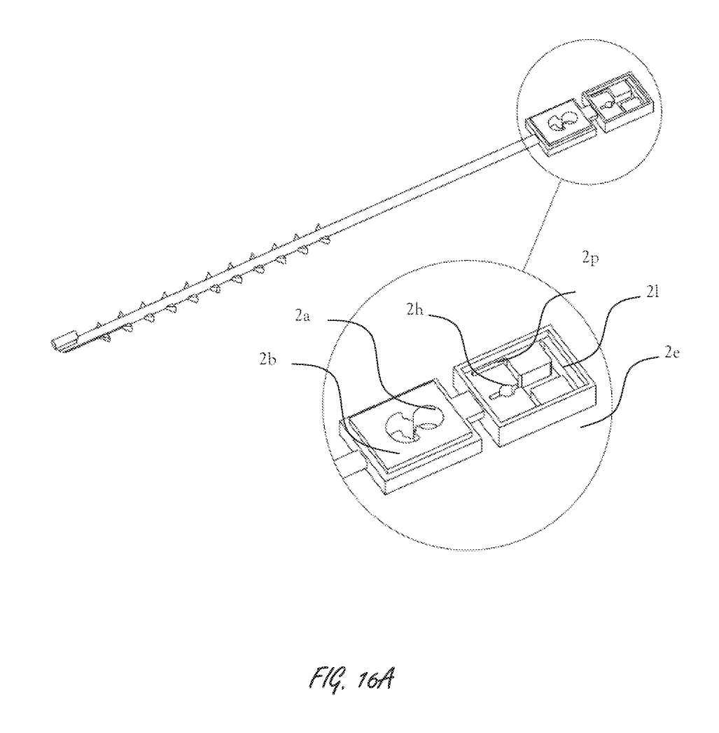

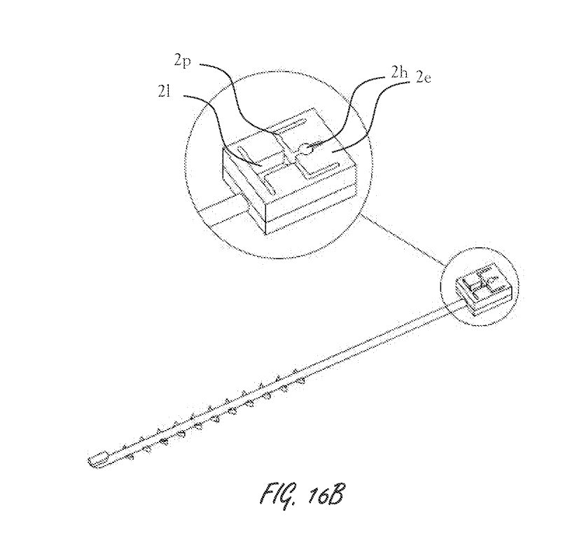

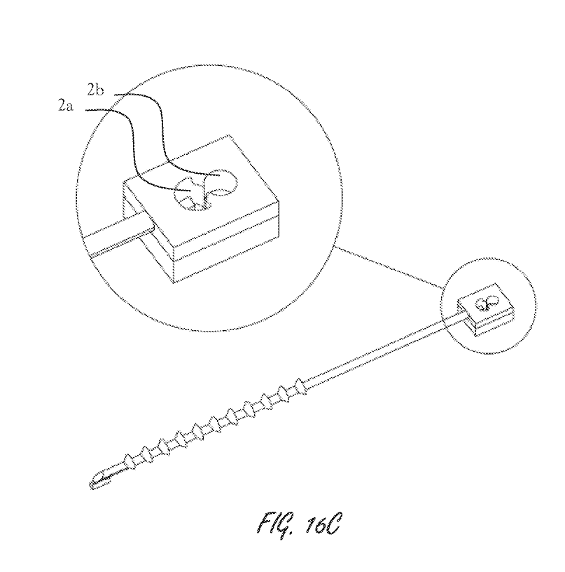

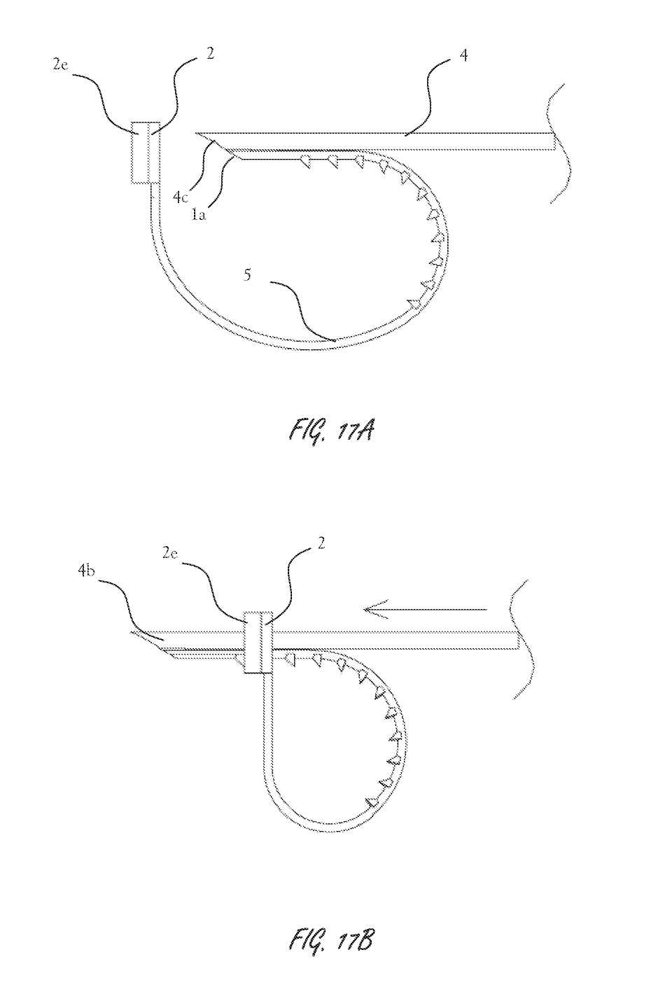

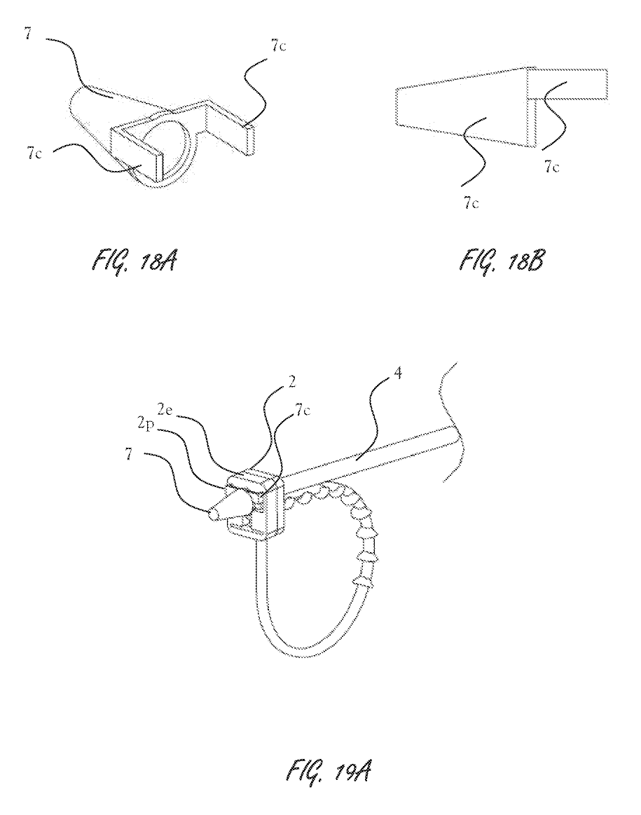

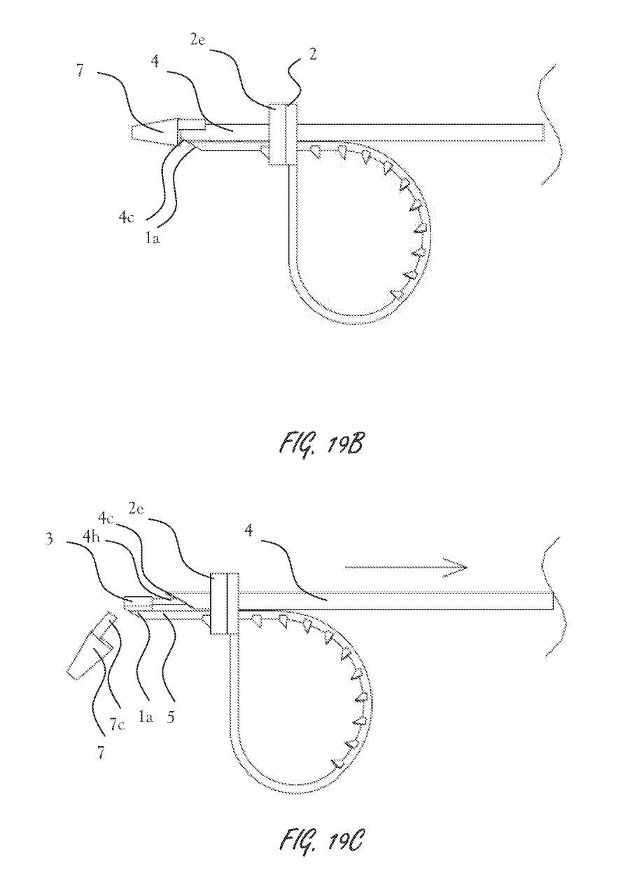

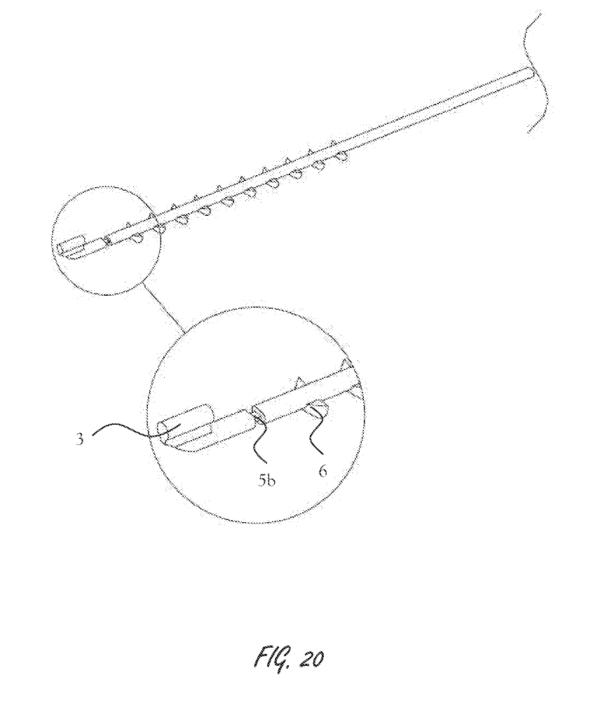

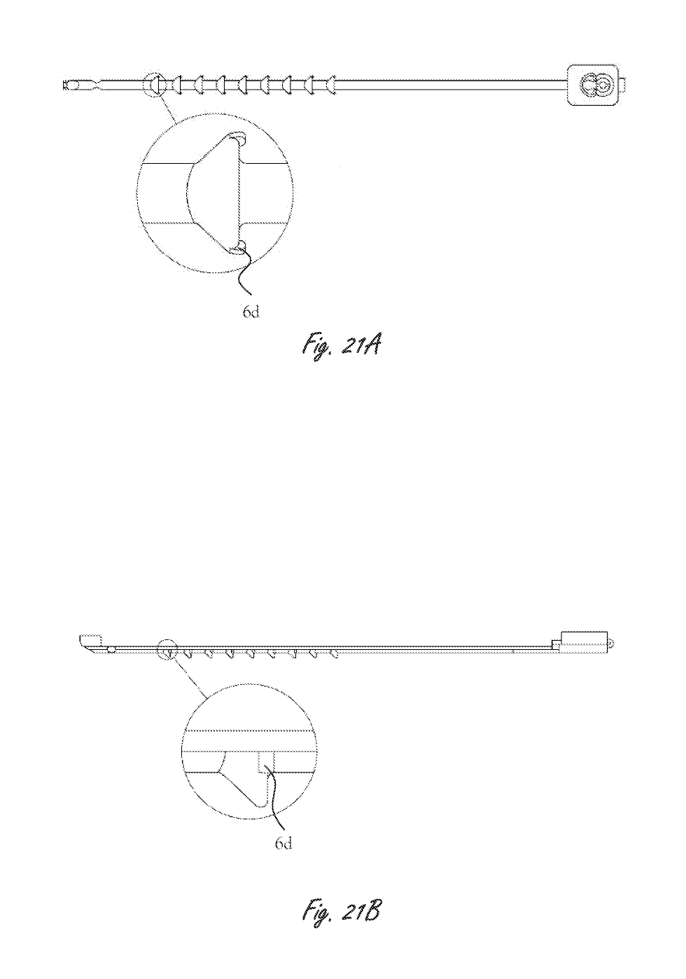

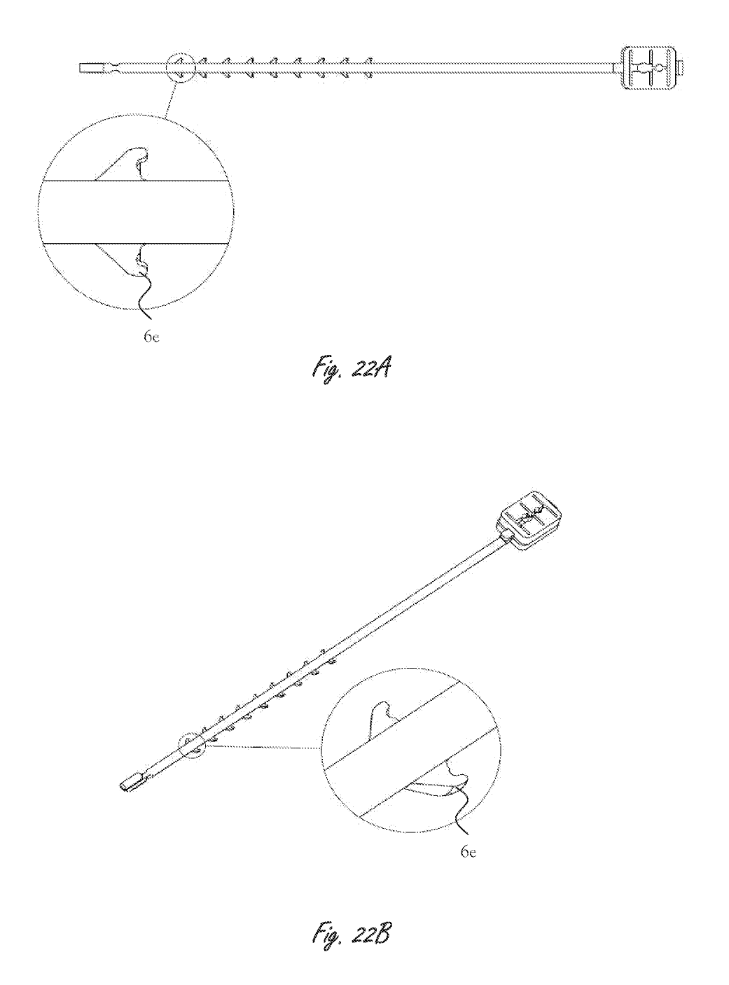

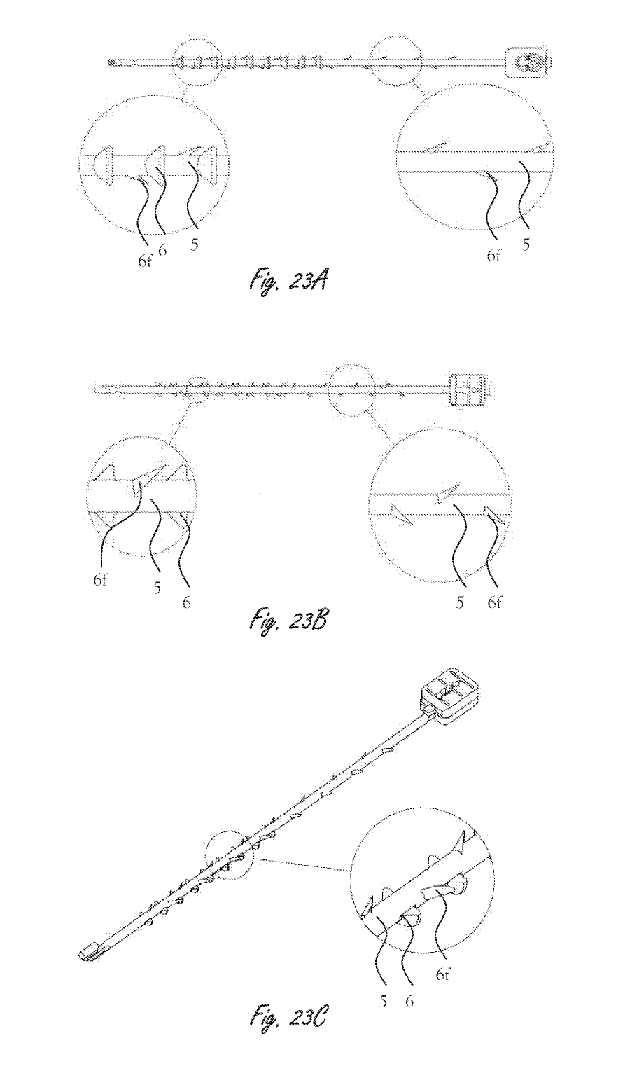

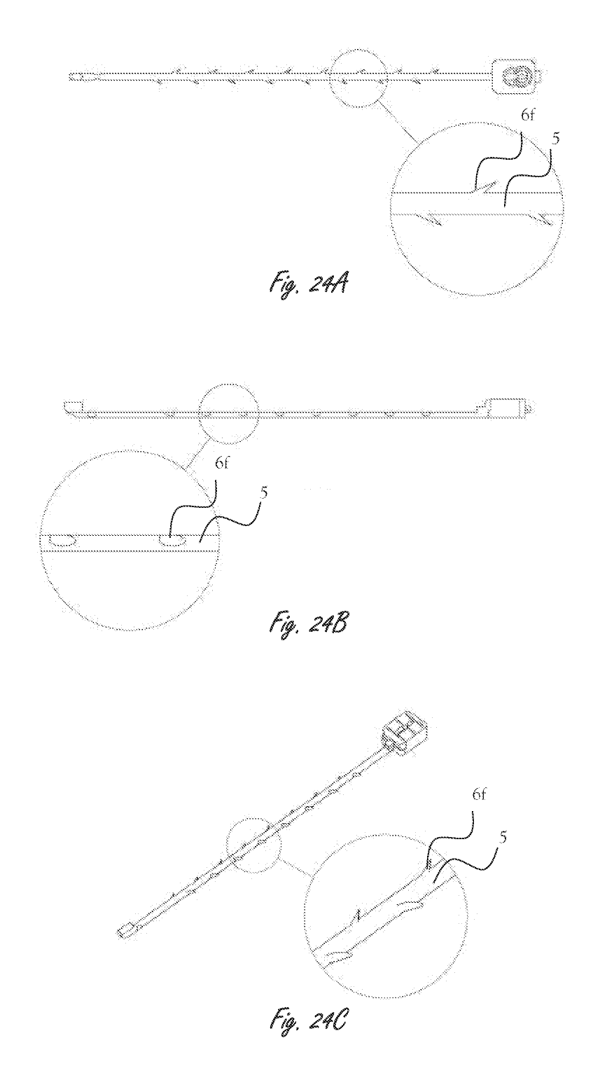

Reference is now made to FIG. 2 which is a schematic drawing of the shaft-pushed fastenable suture (100) partially and temporarily connected to the pushing-shaft (4), where the male-head (1) is being pushed by the pushing-shaft (4) toward the female-head (2). Further, FIG. 2 demonstrates another embodiment where the accepting-channels (2a, 2b), as in FIG. 1, are joined to a single channel (2f).

Reference is now made to FIG. 3 which is a schematic drawing of the shaft-pushed fastenable suture (100) connected to the pushing-shaft (4), where the male-head (1) and the sutures' cord (5) are passing within the female-head (2) accepting channels (2a, 2b).

Reference is now made to FIG. 4 which is a schematic drawing of the pushing-shaft (4) distal end (4b) comprising a sharp portion (4c) with an open bore (4d). The sharp portion (4c) comprises: a tip (4f) and a single notch (4g) both located in opposing directions; therefore the notch (4g) is confined so as to be located on the open bore (4d), opposite to the tip (4f). The bore (4d) and the notch (4g) are adapted to temporarily accommodate the suture's anchor (3). In another embodiment of the present invention the dimensions of the anchor (3) are greater than the dimensions of the notch (4g), such that the anchor (3) is retained by the notch (4g) inside the open bore (4d), so as to prevent movement of the fastenable suture (100) relative to the pushing shaft (4).

Reference is now made to FIGS. 5A and 5B which are schematic drawings demonstrating another embodiment for the present invention where the suture's cord (5) is having a crescent-like cross-section (5a), configured to be at least partially attached with the pushing-shaft (4), for a small tissue's puncture and a smooth passage through the first and second tissues. FIG. 5A shows a side view of the attached crescent-like cord (5a) and pushing-shaft (4) denoting the cross-section “A-A”, which is demonstrated in FIG. 5B. FIG. 5B is the schematic cross-section “A-A” of the attached crescent-like cord (5a) and the pushing-shaft (4). The size of the pushing-shaft (4) and suture's cord (5) are therefore carefully matched to ensure that the pushing-shaft (4) or the suture's cord (5) do not tear tissue and prevent fluid leakage.

Reference is now made to FIGS. 6A and 6B which are schematic drawings demonstrating another embodiment for the present invention where the pushing-shaft (4) is having a crescent-like cross-section (4a), configured to be at least partially attached with the suture's cord (5), for a small tissue's puncture and a smooth passage through the first and second tissues. FIG. 6A shows a side view of the attached crescent-like pushing-shaft (4a) and suture's cord (5) denoting the cross-section “B-B”, which is demonstrated in FIG. 6B. FIG. 6B is the schematic cross-section “B-B” of the attached crescent-like pushing-shaft (4a) and the suture's cord (5). The size of the pushing-shaft (4) and suture's cord (5) are therefore carefully matched to ensure that the pushing-shaft (4) or the suture's cord (5) do not tear tissue and prevent fluid leakage.

Reference is now made to FIGS. 7A and 7B which are schematic drawings demonstrating another embodiment for the present invention where the pushing-shaft (4) and the suture's cord (5) both having a crescent-like cross-section (4a, 5a), configured for a small tissue's puncture and a smooth passage through the first and second tissues. FIG. 7A shows a side view of the attached crescent-like pushing-shaft (4a) and suture's cord (5a) denoting the cross-section “C-C”, which is demonstrated in FIG. 7B. FIG. 7B is the schematic cross-section “C-C” of the attached crescent-like pushing-shaft (4a) and suture's cord (5a). The size of the pushing-shaft (4) and suture's cord (5) are therefore carefully matched to ensure that the pushing-shaft (4) or the suture's cord (5) do not tear tissue and prevent fluid leakage.

Reference is now made to FIGS. 8A and 8B which are schematic drawings demonstrating another embodiment for the present invention where the suture's teeth (6) are having conic structure (6a). The conic structure (6a) starts from the distal edge (6b) of the teeth (6) and expands towards the proximal edge (6c) of same, as shown in FIG. 8A. The conic teeth (6a) are configured for a smooth passage through the first and second tissues and for preventing the backward motion through the suture's accepting-channel (2a), such that the first and second tissues are protected from being ruptured or torn by the teeth (6). FIG. 8B is the schematic cross-section “D-D” of the cord (5) demonstrating a side view of the conic tooth (6a).

Reference is now made to FIG. 9 which is a schematic drawing of another embodiment for the shaft-pushed fastenable suture (100) where the female-head (2) is having conic structure for the accepting channels (2i, 2j). FIGS. 10A and 10B demonstrate top and side views, respectively, for the above mentioned shaft-pushed fastenable suture (100) with the conic accepting channels (2i, 2j).

Reference is now made to FIGS. 11A, 11B, 11C, 11D and 11E which are schematic drawings of the fastenable suture (100), comprising the conic accepting channels (2i, 2j). FIGS. 11A-11E demonstrate the fastenable suture (100) being pushed by the pushing shaft (4), shown in five stages: FIG. 11A demonstrates the approaching distal end (4b) of the pushing-shaft (4) together with the suture's male-head (1) towards the suture's female-head (2); FIG. 11B demonstrates the distal end (4b) of the pushing-shaft (4) together with the sutures' male-head (1) entering into the suture's female-head (2); FIG. 11C demonstrates the fastening of the first distal tooth (6); FIG. 11D demonstrates the retracing of the pushing-shaft back and out of the female-head (2), while the fastenable-suture (100) is fixed by the fastened tooth (6); and FIG. 11E demonstrates the detaching of the pushing-shaft (4) from the fastenable suture (100).

FIGS. 11C, 11D and 11E further demonstrate another embodiment for the fastenable suture (100) where the pushing shaft (4) further comprises a disengaging-rod (4h) threaded through the central axis of the pushing-shaft (4), configured for relative motion inside the pushing-shaft (4) in order to disengage the anchor (3) out of the pushing-shaft's (4) bore (4d) and notch (4g). When the pushing-shaft (4) is retracing backward from the female-head, as in FIG. 11D, the disengaging-rod (4h) stands still preventing the anchor's (3) movement. When anchor (3) is detached from the distal end (4b) of the pushing-shaft (4) the disengaging-rod (4h) retracts out of the female-head (2) joining the retracted pushing-shaft (4), as shown in FIG. 11E. FIG. 11C further demonstrates another embodiment where the anchor (3) comprises a rod-bore (3a) configured to temporarily accommodate the disengaging-rod (4h).

FIGS. 11A and 11D further demonstrate other embodiments for the present invention where the pushing-shaft (4) distal head comprises the sharp portion (4c) as described above and where the male-head (1) comprises a sharp-tip (1a), both configured for smooth passage through the first and second tissue.

Reference is now made to FIG. 12 which is a schematic drawing of the suture's female-head (2) where the suture accepting-channel (2a) and the neighboring pushing-shaft accepting-channel (2b) have a conic shape (2i, 2j) configured to centralize the approaching the male-head (1) and distal end (4b) of the pushing-shaft (4) into the female-head (2). Further, FIG. 12 demonstrates another embodiment for the flexible pawls (2c) which are configured by at least one notch (2d), passing through the conic suture accepting-channel (2i).

Reference is now made to FIGS. 13 and 14 which are schematic drawing of the suture's female-head (2) further comprising a detachable protecting-cap (7) located at the outlet (2k) of the female-head (2) pushing-shaft accepting-channel (2b). The protecting-cap (7), as shown in FIG. 13, is configured to cover the pushing-shaft (4) distal end (4b) when passing through same, such that any other tissues and adjacent blood vessels are protected from being ruptured or torn by the pushing-shaft (4) distal end (4b). In another embodiment, the protecting-cap (7) is configured to cover the pushing-shaft (4) distal end (4b) and the suture's male head (1), when passing through same. The protecting-cap (7) is temporarily attached according to another embodiment of the present invention to the outlet (2k) of the pushing-shaft accepting-channel (2b), by fasteners (7a) or by a connecting tear-strip (not shown) between the outlet (2k) and the protecting-cap (7). FIG. 14 is a schematic side view of the fastenable suture (100) comprising the detachable protecting-cap (7). FIG. 14 further demonstrates the male-head (1) a sharp-tip (1a), as mentioned above, configured for smooth passage through the first and second tissue.

Reference is now made to FIGS. 15A, 15B and 15C which are three schematic demonstrations of the pushing-shaft (4) pushing and detaching the protecting-cap (7), fastening the first sutures tooth (6) and retracing from the fastenable suture (100), respectively. When the pushing-shaft (4) is pushing the male-head (1) into the female-head (2), the pushing-shaft (4) distal-end (4b) get into the protecting-cap (7), as shown in FIG. 15A, such that the protecting-cap (7) is detached from the female-head (2) outlet (2k), as in FIG. 15B. When the pushing-shaft (4) is retracing out of the female-head (2), the protecting-cap (7) is being blocked by the female-head (2) and falls off and away from distal-end (4b) of the pushing-shaft (4), as shown in FIG. 15C. It is therefore a preferred embodiment of the present invention that the protecting-cap (7) is made of an absorbable material.

Reference is now made to FIGS. 16A, 16B and 16C which are schematic drawings of another embodiment for the present invention where the suture's female-head (2) in connection with the flexible pawls (2c) configured as a flexible flap-section (2e) having line-notches (2l), pin-notches (2p) and a round-notch (2h) for the passage of the pushing-shaft (4), shown in two positions: open, as in FIG. 16A, and flipped towards the female-head (2), as shown in FIG. 16B for top view and FIG. 16C for bottom view. The flexible flap-section (2e) is configured to flip and fold toward the female-head (2), such that the pushing-shaft (4) and the fastenable suture (100) pass through both the accepting channels (2a, 2b) and the flap-section (2e).

According to a preferred embodiment of the present invention the shaft-pushed fastenable suture (100) can be manufactured as one piece including the flexible flap-section (2e). In yet another embodiment the shaft-pushed fastenable suture (100) can manufactured as one piece including the flexible flap-section (2e) and the protecting cap (7).

Reference is now made to FIGS. 17A and 17B which are schematic demonstrations of the pushing-shaft (4) pushing the suture (100) towards the closed flexible flap-section (2e), FIG. 17A, and into the closed flexible flap-section (2e) and fastening the first distal tooth (6), FIG. 17B.

Reference is now made to FIGS. 18A and 18B which are two schematic views of another embodiment for the detachable protecting-cap (7). In this embodiment the protecting-cap (7) is having at least one pin (7c) configured to be accommodated by pin-notches (2p) of the flexible flap-section (2e).

Reference is now made to FIGS. 19A, 19B and 19C which are schematic demonstrations of the pushing-shaft (4) pushing the male-head (1) into the female-head (2) with the protecting-cap (7) having the two pins (7c). FIG. 19A demonstrates that ones the distal-end (4) of the pushing shaft enters into the suture's female-head (2) the sharp portion (4c) is covered by the protecting-cap (7). FIG. 19B demonstrates that as the pushing-shaft (4) continues forward through the female-head (2) the protecting-cap (7) pins (7c) are being pushed out of their accommodating pin-notches (2p); FIG. 19B further demonstrates the fastening of the first distal tooth (6). FIG. 19C demonstrates that when the pushing-shaft (4) is retracing out of the female-head (2), the protecting-cap (7) is being blocked by the female-head (2) and falls off and away from distal-end (4b) of the pushing-shaft (4), thereby the distal end (4b) of a pushing-shaft (4) is detached from the protecting-cap (7).

FIGS. 19B and 19C further demonstrate the above mentioned embodiments for the present invention where the pushing-shaft (4) distal head comprises the sharp portion (4c) as described above and where the male-head (1) comprises a sharp-tip (1a), both configured for smooth passage through the first and second tissue.

Reference is now made to FIG. 20 demonstrating a schematic drawing of another embodiment for the present invention, where the suture's cord (5) comprises a weakened section (5b) which is narrower than the rest of the cord (5), located between the anchor (3) and the first distal tooth (6), configured to be ripped for detaching the anchor (3) and thereby the pushing-shaft (4) from the cord (5), when the cord's (5) tensile tension has reached a predetermined tension. This configuration is set for avoiding unnecessary or even harmful stress to the first and second tissues. In a preferred embodiment the thickness of the weakened section (5b) is set to be ripped at predetermined tension of about 400 gram-force.

Reference is now made to FIGS. 21A and 21B which are schematic upper and side views of another embodiment of the present invention, where at least one of the teeth (6) comprises at least one protruding flapping-tip (6d), at the proximal edge (6c), configured to be pressed towards the cord (5) when the cord (5) is being pushed forward via the first and second tissues and via the female-head (2), and further configured to protrude away from the cord (5) and against the first and second tissues and/or against the female-head (2), when the cord (5) is being pulled backwards, such that the protruding flapping tip (6d) assist in fastening the teeth (6) to the female-head (2) and/or to the first and second tissues and assist in fixating and fastening the suture. According to another embodiment, the protruding flapping-tip (6d) can be at least partially flexible. According to another embodiment, the protruding flapping-tip (6d) can be revolving.

Reference is now made to FIG. 22A and 22B which are schematic bottom and bottom-isometric views of another embodiment of the present invention, where at least one of the teeth (6) comprises a flexible flap (6e), at the proximal edge (6c), configured to be pressed towards the cord (5) when the cord is being pushed forward via the first and second tissue and/or via the female-head (2), and further configured to protrude away from the cord (5), when the cord (5) is being pulled backwards against the first and second tissue and/or the female-head (2), such that the flexible flap (6e) assist in fastening the teeth (6) to the female-head (2) and/or to the first and second tissues and assist in fixating and fastening the suture.

Reference is now made to FIGS. 23A, 23B and 23C which are schematic upper, bottom and bottom-isometric views of another embodiment of the present invention, where the cord (5) comprises one or more backward facing barbs (6f), configured allow forward motion of the cord (5), when the cord is pushed forward via the first and second tissues and via the female-head (2), and when the cord is being pulled backwards the barbs (6f) are configured to stick against the first and second tissues and/or against the female-head (2), such that the barbs (6f) assist in fixating and fastening the suture.

According to another embodiment, the one or more teeth (6) are replaced by, or alternatively configured as, backward facing barb (6f), as demonstrated in FIGS. 24A, 24B and 24C (upper, side and bottom-isometric views), configured to allow forward motion of the cord (5) when the cord (6) being is pushed forward via the first and second tissues and via the female-head (2), and when the cord is being pulled backwards the barbs (6f) are configured to stick and/or stab against the first and second tissues and/or against the female-head (2), such that the barbs (6f) are fixating and fastening the suture.

According to another embodiment, the backward facing barbs (6f) mentioned above are circumferentially distributed for spreading tension. According to another embodiment the circumferential distribution is helical and according to another embodiment the circumferential distribution is evenly spaced.

In surgery or medical procedure, a ligature or a ligation system consists of a piece of thread (suture) or a polymer clip, such as the Hem-O-Lok®, tied around an anatomical structure, usually a blood vessel or another hollow structure (e.g. urethra, uterus, stomach, esophagus, small intestine, colon) in order to occlude it. Usually the surgeon clamps the vessel perpendicular to the axis of the artery or vein with a hemostat, then secures it by ligaturing; i.e. using a Hem-O-Lok® around the artery or vein before releasing the hemostat.

According to another embodiment, the present invention further provides the new shaft-pushed fastenable suture (100) for ligation of at least two blood vessels.

According to another embodiment, the present invention further provides the new shaft-pushed fastenable suture (100) for ligation and at least partial occlusion of at least one blood vessel and/or a hollow organ (e.g. urethra, uterus, stomach, esophagus, small intestine, or colon).

The advantages of such ligation with the above mentioned new fastenable suture (100) are:
the fastenable suture (100) can be secured by and tightened to a chosen measure, set by the motion of the pushing-shaft, thereby allowing the gathering of blood vessels with or without their occlusion;
since the fastenable suture (100) can be secured by and tightened to a chosen measure, it is less “sensitive” to the angle of its placement or application;
the fastenable suture (100) can be inserted into the body via smaller entry ports (3-5 mm), where other solutions requires larger entry ports (10-12 mm), for the entrance of their applier; and
when passing through or puncturing a tissue, it is the sharp pushing-shaft (4) of the fastenable suture that conducts the cutting and passing through, rather than the polymer tip as in the application of the Hem-O-Lok®; thereby the sharp pushing-shaft ensures minimal tissue's puncture with no damage as the suture is drawn through.

The present invention being thus described in terms of several embodiments and examples, it will be appreciated that the same may be varied in many ways. Such variations are not to be regarded as a departure from the spirit and scope of the invention, and all such modifications as would be obvious to one skilled in the art are contemplated.

1-77. (canceled)

78. A shaft-pushed fastenable suture (100) for suturing a first tissue with a second tissue, characterized by an elongated flexible cord (5), with one or more teeth (6) protruding from same, having a male-head (1) at the distal end and an accepting female-head (2) at the proximal end; said male-head (1) is in connection with an anchor (3); said anchor configured to be temporarily accommodated by a distal end (4b) of a pushing-shaft (4); said female-head (2) comprising a suture accepting-channel (2a) and a neighboring pushing-shaft accepting-channel (2b); at least one of said two accepting-channels (2a, 2b) comprising one or more flexible pawls (2c) for both allowing, by means of said teeth (6), a continuous linear forward motion of said male-head (1) and said cord (5) within said accepting-channel (2a, 2b) in only one direction, while preventing motion of said male-head (1) and cord (5) in a backward direction and allowing only said pushing-shaft (4) a reversible reciprocal motion.

79. The shaft-pushed fastenable suture (100) according to claim 78, wherein said cord (5) having a crescent-like (5a) cross-section (A-A), configured to be at least partially attached with said pushing-shaft (4), for a small puncture and a smooth passage through said first and second tissues.

80. The shaft-pushed fastenable suture (100) according to claim 78, wherein said pushing-shaft (4) having a crescent-like (4a) cross-section (B-B) configured to be at least partially attached with said cord (5), for a small puncture and a smooth passage through said first and second tissues.

81. The shaft-pushed fastenable suture (100) according to claim 78, wherein said one or more teeth (6) having a conic structure (6a), starting from the distal edge (6b) of said teeth (6) and expanding towards the proximal edge (6c) of same, configured for a smooth passage through said first and second tissues and for preventing said backward motion through said suture accepting-channel (2a) such that said first and second tissues are protected from being ruptured or torn by said teeth (6).

82. The shaft-pushed fastenable suture (100) according to claim 78, wherein said male-head (1) comprises a sharp tip (1a) configured to be pushed by said pushing-shaft (4) through said first and second tissues.

83. The shaft-pushed fastenable suture (100) according to claim 78, wherein said female-head (2) configuration together with said pushing-shaft distal end (4b) are adapted to create tensile-tension to said first and second tissues, as said pushing-shaft distal end (4b) approaches said female-head (2), such that the friction of said suture (100) and said pushing-shaft (4) with said first and second tissues is reduced, a minimal puncture is obtained and tears, ruptures or organ blockage are avoided.

84. The shaft-pushed fastenable suture (100) according to claim 83, wherein said pushing-shaft distal end (4b) is of blunt contour (4i).

85. The shaft-pushed fastenable suture (100) according to claim 78, wherein said suture accepting-channel (2a) and said neighboring pushing-shaft accepting-channel (2b) having a conic structure (2i, 2j), configured to centralize the approaching said male-head (1) and said distal end (4b) of said pushing-shaft (4) into said female-head (2).

86. The shaft-pushed fastenable suture (100) according to claim 85, wherein said one or more flexible pawls (2c) are configured by at least one notch (2d) passing through said conic suture accepting-channel (2i).

87. The shaft-pushed fastenable suture (100) according to claim 78, wherein said one or more flexible pawls (2c) are configured as a flexible flap-section (2e) having at least one line-notch (21); said flap-section (2e) is in connection with said female-head (2), configured to flip and fold toward said female-head (2), such that said pushing-shaft (4) and said suture (100) pass through both said accepting channels (2a, 2b) and said flap-section (2e).

88. The shaft-pushed fastenable suture (100) according to claim 87, wherein said flap-section (2e) further comprising a round-notch (2h) configured for the passage of said pushing-shaft (4).

89. The shaft-pushed fastenable suture (100) according to claim 78, wherein said accepting-channels (2a, 2b) are joined to a single channel (2f).

90. The shaft-pushed fastenable suture (100) according to claim 78, wherein said female-head (2) further comprising a detachable protecting-cap (7) at the outlet (2k) of said pushing-shaft accepting-channel (2b), configured to cover at least one selected from a group consisting of: said distal end (4b) of said pushing-shaft (4) and said male head (1), when passing through same, such that any other tissues and adjacent blood vessels are protected from being ruptured or torn.

91. The shaft-pushed fastenable suture (100) according to claim 90, wherein said protecting-cap (7) is temporarily attached to said outlet (2k) of said pushing-shaft accepting-channel (2b), by pressure-fasteners (7a) or by a connecting tear-strip between said outlet (2k) and said protecting-cap (7).

92. The shaft-pushed fastenable suture (100) according to claim 78, wherein said pushing-shaft (4) distal end (4b) comprising a sharp portion (4c) having an open bore (4d);
said sharp portion (4c) comprises: a tip (4f) and a single notch (4g); said notch (4g) confined so as to be located on said open bore (4d), opposite to said tip (4f); said bore (4d) and said notch (4g) are adapted to accommodate said anchor (3).

93. The shaft-pushed fastenable suture (100) according to claim 92, wherein the dimensions of said anchor (3) are greater than the dimensions of said notch (4g), such that said anchor (3) is retained by said notch (4g) inside said open bore (4d), so as to prevent movement of said suture (100) relative to said pushing shaft (4).

94. The shaft-pushed fastenable suture (100) according to claim 87, wherein said shaft-pushed fastenable suture (100) is manufactured as one piece, such that said flexible flap-section (2e) is configured to flip and fold toward said female-head (2).

95. The shaft-pushed fastenable suture (100) according to claim 90, wherein said shaft-pushed fastenable suture (100) is manufactured as one piece, such that said protecting-cap (7) is configured to flip and fold toward said outlet (2k) of said female-head (2).

96. The shaft-pushed fastenable suture (100) according to claim 78, wherein said pushing-shaft (4) comprises a disengaging-rod (4h) threaded through the center axis of said pushing-shaft (4) and configured for relative motion inside said pushing-shaft (4) for separating and disengaging said anchor (3) from said pushing shaft (4) and releasing said fastenable suture (100) from said pushing-shaft (4).

97. The shaft-pushed fastenable suture (100) according to claim 96, wherein said anchor (3) comprises a rod-bore (3a) configured to temporarily accommodate said disengaging-rod (4h).

98. The shaft-pushed fastenable suture (100) according to claims 87 and 90 wherein said flexible flap-section (2e) further comprising at least one pin-notch (2p) and wherein said protecting cap (7) comprises at least one pin (7c), configured to be accommodated by said at least one of said pin-notches (2p).

99. The shaft-pushed fastenable suture (100) according to claim 78, wherein the suture's cord (5) comprises a weakened section (5b), which is narrower than said cord (5), located between said anchor (3) and the first adjacent said tooth (6), configured to be ripped and detached form said anchor (3), when said cord's (5) tensile tension has reached a predetermined tensile-tension.

100. The shaft-pushed fastenable suture (100) according to claim 78, wherein said fastenable suture (100) is configured for self-tying and self-fastening facilitating a fully automatic suturing.

101. The shaft-pushed fastenable suture (100) according to claim 78, wherein at least two of said fastenable sutures (100) and their accompanying said pushing shafts (4) are configured to be mounted on an automatic surgical suturing device.

102. The shaft-pushed fastenable suture (100) according to claim 78, wherein at least one of said one or more teeth (6) comprises at least one protruding flapping-tip (6d), at said proximal edge (6c).

103. The shaft-pushed fastenable suture (100) according to claim 102, wherein said at least one protruding flapping-tip (6d), is at least partially flexible.

104. The shaft-pushed fastenable suture (100) according to claim 102, wherein said at least one protruding flapping-tip (6d), is revolving toward and away from said cord (5).

105. The shaft-pushed fastenable suture (100) according to claim 78, wherein at least one of said one or more teeth (6) comprises a flap (6e), at said proximal edge (6c).

106. The shaft-pushed fastenable suture (100) according to claim 105, wherein said flap (6e), is at least partially flexible.

107. The shaft-pushed fastenable suture (100) according to claim 78, wherein said cord (5) comprises one or more backward facing barbs (6f).

108. The shaft-pushed fastenable suture (100) according to claim 78, wherein said one or more teeth (6) are configured as backward facing barbs (6f).

109. The shaft-pushed fastenable suture (100) according to claim 107 or 108, wherein said barbs (6f) are at least partially flexible.

110. The shaft-pushed fastenable suture (100) according to claim 107 or 108, wherein said barbs (6f) are evenly spread.

111. The shaft-pushed fastenable suture (100) according to claim 107 or 108, wherein said barbs (6f) are circumferentially distributed.

112. The shaft-pushed fastenable suture (100) according to claim 107 or 108, wherein said barbs (6f) are helically distributed.

113. The shaft-pushed fastenable suture (100) according to claim 78, wherein said suture (100) further configured for ligation of at least two blood vessels.

114. The shaft-pushed fastenable suture (100) according to claim 78, wherein said suture (100) further configured for ligation and at least partial occlusion of at least one blood vessel and/or hollow structured organ.

115. A shaft-pushed fastenable suture (100) for ligation of at least one blood vessels and/or hollow structured organ, characterized by an elongated flexible cord (5), with one or more teeth (6) protruding from same, having a male-head (1) at the distal end and an accepting female-head (2) at the proximal end; said male-head (1) is in connection with an anchor (3); said anchor configured to be temporarily accommodated by a distal end (4b) of a pushing-shaft (4); said female-head (2) comprising a suture accepting-channel (2a) and a neighboring pushing-shaft accepting-channel (2b); at least one of said two accepting-channels (2a, 2b) comprising one or more flexible pawls (2c) for both allowing, by means of said teeth (6), a continuous linear forward motion of said male-head (1) and said cord (5) within said accepting-channel (2a, 2b) in only one direction, while preventing motion of said male-head (1) and cord (5) in a backward direction and allowing only said pushing-shaft (4) a reversible reciprocal motion.
